SEQUENCE LISTING (37 sequences)

SEQ ID NO: 1 - protein, 12 aa, Artificial Sequence
  Synthetic peptide
CXXXXXXXXCXC

SEQ ID NO: 2 - protein, 12 aa, Artificial Sequence
  Synthetic peptide
CXDXXXXYXCXC

SEQ ID NO: 3 - protein, 12 aa, Artificial Sequence
  Synthetic peptide
CXDXXXXFXCXC

SEQ ID NO: 4 - protein, 12 aa, Artificial Sequence
  Synthetic peptide
CXNXXXXYXCXC

SEQ ID NO: 5 - protein, 12 aa, Artificial Sequence
  Synthetic peptide
CXNXXXXFXCXC

SEQ ID NO: 6 - protein, 12 aa, Artificial Sequence
  Synthetic peptide
CTXLVANXSCEC

SEQ ID NO: 7 - protein, 12 aa, Artificial Sequence
  Synthetic peptide
CKXDISSXECWC

SEQ ID NO: 8 - protein, 12 aa, Mus musculus
CTDLVANYSCEC

SEQ ID NO: 9 - DNA, 2303 bp, Mus musculus
  CDS (619)..(2061)
gaattccggttaactgaggacaaagggtaatgcagaagtgatatttgatttccattctcattcccagtggccttgatatttaaactgattcctgccaccaggtccttgggccaccctgtccctgcgtctcatatttctgcatgctgctttgtttgtatatagtgcgctcctggcctcaggctcgctcccctccagctctcgcttcattgttctccaagtcagaagcccccgcatccgccgcgcagcagcgtgagccgtagtcactgctggccgcttcgcctgcgtgcgcgcacggaaatcggggagccaggaacccaaggagccgccgtccgcccgctgtgcctctgctagaccactcgcagccccagcctctctcaagcgcacccacctccgcgcaccccagctcaggcgaagctggagtgagggtgaatcaccctttctctagggccaccactcttttatcgcccttcccaagatttgagaagcgctgcgggaggaaagacgtcctcttgatctctgacagggcggggtttactgctgtcctgcaggcgcgcctcgcctactgtgccctccgctacgaccccggaccagcccaggtcacgtccgtgagaagggatcatgaagcacttggtagcagcctggcttttggttggactcagcctcggggtgccccagttcggcaaaggtgacatttgcaacccgaacccctgtgaaaatggtggcatctgtctgtcaggactggctgatgattccttttcctgtgagtgtccagaaggcttcgcaggtccgaactgctctagtgttgtggaggttgcatcagatgaagaaaagcctacttcagcaggtccctgcatccctaacccatgccataacggaggaacctgtgagataagcgaagcctatcgaggagacacattcataggctatgtttgtaaatgtcctcggggatttaatgggattcactgtcagcacaatataaatgaatgtgaagctgagccttgcagaaatggcggaatatgtaccgaccttgttgctaactactcttgtgaatgcccaggagaatttatgggacgaaattgtcaatataaatgctctgggccattgggaatcgaaggtgggatcatatctaatcagcaaatcacagcttcatctactcaccgagctctttttggactccggaagtggtatccctactatgctcgacttaataagaagggccttataaatgcctggacagctgctgaaaatgacagatggccatggattcagataaatttgcaaagaaaaatgagagtcactggtgttattacccaaggagcaaaaaggattggaagcccagagtacataaaatcctacaaaattgcctacagcaatgacgggaagacctgggcaatgtacaaagtaaaaggcaccaatgaagagatggtctttcgtggaaatgttgataacaacacaccatatgctaattctttcacacccccaatcaaagctcagtatgtaagactctacccccaaatttgtcgaaggcattgtactttaagaatggaacttcttggctgtgagctctcaggctgttcagaacctttggggatgaaatcagggcatatacaagactaccagatcactgcctccagcgtcttcagaacactcaacatggacatgtttacttgggaaccaaggaaagccaggctggacaagcaaggcaaagtaaatgcctggacttccggccataacgaccagtcacaatggttacaggttgatcttcttgtccctactaaggtgacaggcatcattacacaaggagctaaagattttggtcacgtgcagtttgttgggtcatacaaactagcttacagcaatgatggagaacactggatggtgcaccaggatgaaaaacagaggaaagacaaggtttttcaaggcaattttgacaatgacactcacaggaaaaatgtcatcgaccctcccatctatgcacgattcataagaatccttccttggtcctggtatggaaggatcactctgcggtcagagctgctgggctgcgcagaggaggaatgaagtgcggggccgcacatcccacaatgcttttctttattttcctataagtatctccacgaaatgaactgtgtgaagctgatggaaactgcatttgtttttttcaaagtgttcaaattatggtaggctactgactgtctttttaggagttctaagcttgcctttttaataatttaatttggtttcctttgctcaactctcttatgtaatatcacactgtctgtgagttactcttcttgttctct

SEQ ID NO: 10 - protein, 480 aa, Mus musculus
MKHLVAAWLLVGLSLGVPQFGKGDICNPNPCENGGICLSGLADDSFSCECPEGFAGPNCSSVVEVASDEEKPTSAGPCIPNPCHNGGTCEISEAYRGDTFIGYVCKCPRGFNGIHCQHNINECEAEPCRNGGICTDLVANYSCECPGEFMGRNCQYKCSGPLGIEGGIISNQQITASSTHRALFGLRKWYPYYARLNKKGLINAWTAAENDRWPWIQINLQRKMRVTGVITQGAKRIGSPEYIKSYKIAYSNDGKTWAMYKVKGTNEEMVFRGNVDNNTPYANSFTPPIKAQYVRLYPQICRRHCTLRMELLGCELSGCSEPLGMKSGHIQDYQITASSVFRTLNMDMFTWEPRKARLDKQGKVNAWTSGHNDQSQWLQVDLLVPTKVTGIITQGAKDFGHVQFVGSYKLAYSNDGEHWMVHQDEKQRKDKVFQGNFDNDTHRKNVIDPPIYARFIRILPWSWYGRITLRSELLGCAEEE

SEQ ID NO: 11 - DNA, 105 bp, Mus musculus
  CDS (1)..(105)
tgtgaagctgagccttgcagaaatggcggaatatgtaccgaccttgttgctaactactcttgtgaatgcccaggagaatttatgggacgaaattgtcaatataaa

SEQ ID NO: 12 - protein, 35 aa, Mus musculus
CEAEPCRNGGICTDLVANYSCECPGEFMGRNCQYK

SEQ ID NO: 13 - DNA, 1044 bp, Mus musculus
  CDS (1)..(1044)
atgaagcacttggtagcagcctggcttttggttggactcagcctcggggtgccccagttcggcaaaggtgacatttgcaacccgaacccctgtgaaaatggtggcatctgtctgtcaggactggctgatgattccttttcctgtgagtgtccagaaggcttcgcaggtccgaactgctctagtgttgtggaggttgcatcagatgaagaaaagcctacttcagcaggtccctgcatccctaacccatgccataacggaggaacctgtgagataagcgaagcctatcgaggagacacattcataggctatgtttgtaaatgtcctcggggatttaatgggattcactgtcagcacaatataaatgaatgtgaagctgagccttgcagaaatggcggaatatgtaccgaccttgttgctaactactcttgtgaatgcccaggagaatttatgggacgaaattgtcaatataaatgctctgggccattgggaatcgaaggtgggatcatatctaatcagcaaatcacagcttcatctactcaccgagctctttttggactccggaagtggtatccctactatgctcgacttaataagaagggccttataaatgcctggacagctgctgaaaatgacagatggccatggattcagataaatttgcaaagaaaaatgagagtcactggtgttattacccaaggagcaaaaaggattggaagcccagagtacataaaatcctacaaaattgcctacagcaatgacgggaagacctgggcaatgtacaaagtaaaaggcaccaatgaagagatggtctttcgtggaaatgttgataacaacacaccatatgctaattctttcacacccccaatcaaagctcagtatgtaagactctacccccaaatttgtcgaaggcattgtactttaagaatggaacttcttggctgtgagctctcaggctgttcagaacctttggggatgaaatcagggcatatacaagactaccagatcactgcctccagcgtcttcagaacactcaacatggacatg

SEQ ID NO: 14 - protein, 348 aa, Mus musculus
MKHLVAAWLLVGLSLGVPQFGKGDICNPNPCENGGICLSGLADDSFSCECPEGFAGPNCSSVVEVASDEEKPTSAGPCIPNPCHNGGTCEISEAYRGDTFIGYVCKCPRGFNGIHCQHNINECEAEPCRNGGICTDLVANYSCECPGEFMGRNCQYKCSGPLGIEGGIISNQQITASSTHRALFGLRKWYPYYARLNKKGLINAWTAAENDRWPWIQINLQRKMRVTGVITQGAKRIGSPEYIKSYKIAYSNDGKTWAMYKVKGTNEEMVFRGNVDNNTPYANSFTPPIKAQYVRLYPQICRRHCTLRMELLGCELSGCSEPLGMKSGHIQDYQITASSVFRTLNMDM

SEQ ID NO: 15 - DNA, 1095 bp, Mus musculus
  CDS (1)..(1095)
atgaagcacttggtagcagcctggcttttggttggactcagcctcggggtgccccagttcggcaaaggtgacatttgcaacccgaacccctgtgaaaatggtggcatctgtctgtcaggactggctgatgattccttttcctgtgagtgtccagaaggcttcgcaggtccgaactgctctagtgttgtggaggttgcatcagatgaagaaaagcctacttcagcaggtccctgcatccctaacccatgccataacggaggaacctgtgagataagcgaagcctatcgaggagacacattcataggctatgtttgtaaatgtcctcggggatttaatgggattcactgtcagcacaatataaatgaatgtgaagctgagccttgcagaaatggcggaatatgtaccgaccttgttgctaactactcttgtgaatgcccaggagaatttatgggacgaaattgtcaatataaatgctctgggccattgggaatcgaaggtgggatcatatctaatcagcaaatcacagcttcatctactcaccgagctctttttggactccggaagtggtatccctactatgctcgacttaataagaagggccttataaatgcctggacagctgctgaaaatgacagatggccatggattcagataaatttgcaaagaaaaatgagagtcactggtgttattacccaaggagcaaaaaggattggaagcccagagtacataaaatcctacaaaattgcctacagcaatgacgggaagacctgggcaatgtacaaagtaaaaggcaccaatgaagagatggtctttcgtggaaatgttgataacaacacaccatatgctaattctttcacacccccaatcaaagctcagtatgtaagactctacccccaaatttgtcgaaggcattgtactttaagaatggaacttcttggctgtgagctctcaggctgttcagaacctttggggatgaaatcagggcatatacaagactaccagatcactgcctccagcgtcttcagaacactcaacatggacatgtttacttgggaaccaaggaaagccaggctggacaagcaaggcaaagtaaat

SEQ ID NO: 16 - protein, 365 aa, Mus musculus
MKHLVAAWLLVGLSLGVPQFGKGDICNPNPCENGGICLSGLADDSFSCECPEGFAGPNCSSVVEVASDEEKPTSAGPCIPNPCHNGGTCEISEAYRGDTFIGYVCKCPRGFNGIHCQHNINECEAEPCRNGGICTDLVANYSCECPGEFMGRNCQYKCSGPLGIEGGIISNQQITASSTHRALFGLRKWYPYYARLNKKGLINAWTAAENDRWPWIQINLQRKMRVTGVITQGAKRIGSPEYIKSYKIAYSNDGKTWAMYKVKGTNEEMVFRGNVDNNTPYANSFTPPIKAQYVRLYPQICRRHCTLRMELLGCELSGCSEPLGMKSGHIQDYQITASSVFRTLNMDMFTWEPRKARLDKQGKVN

SEQ ID NO: 17 - DNA, 1104 bp, Mus musculus
  CDS (1)..(1104)
atgaagcacttggtagcagcctggcttttggttggactcagcctcggggtgccccagttcggcaaaggtgacatttgcaacccgaacccctgtgaaaatggtggcatctgtctgtcaggactggctgatgattccttttcctgtgagtgtccagaaggcttcgcaggtccgaactgctctagtgttgtggaggttgcatcagatgaagaaaagcctacttcagcaggtccctgcatccctaacccatgccataacggaggaacctgtgagataagcgaagcctatcgaggagacacattcataggctatgtttgtaaatgtcctcggggatttaatgggattcactgtcagcacaatataaatgaatgtgaagctgagccttgcagaaatggcggaatatgtaccgaccttgttgctaactactcttgtgaatgcccaggagaatttatgggacgaaattgtcaatataaatgctctgggccattgggaatcgaaggtgggatcatatctaatcagcaaatcacagcttcatctactcaccgagctctttttggactccggaagtggtatccctactatgctcgacttaataagaagggccttataaatgcctggacagctgctgaaaatgacagatggccatggattcagataaatttgcaaagaaaaatgagagtcactggtgttattacccaaggagcaaaaaggattggaagcccagagtacataaaatcctacaaaattgcctacagcaatgacgggaagacctgggcaatgtacaaagtaaaaggcaccaatgaagagatggtctttcgtggaaatgttgataacaacacaccatatgctaattctttcacacccccaatcaaagctcagtatgtaagactctacccccaaatttgtcgaaggcattgtactttaagaatggaacttcttggctgtgagctctcaggctgttcagaacctttggggatgaaatcagggcatatacaagactaccagatcactgcctccagcgtcttcagaacactcaacatggacatgtttacttgggaaccaaggaaagccaggctggacaagcaaggcaaagtaaatgcctggact

SEQ ID NO: 18 - protein, 368 aa, Mus musculus
MKHLVAAWLLVGLSLGVPQFGKGDICNPNPCENGGICLSGLADDSFSCECPEGFAGPNCSSVVEVASDEEKPTSAGPCIPNPCHNGGTCEISEAYRGDTFIGYVCKCPRGFNGIHCQHNINECEAEPCRNGGICTDLVANYSCECPGEFMGRNCQYKCSGPLGIEGGIISNQQITASSTHRALFGLRKWYPYYARLNKKGLINAWTAAENDRWPWIQINLQRKMRVTGVITQGAKRIGSPEYIKSYKIAYSNDGKTWAMYKVKGTNEEMVFRGNVDNNTPYANSFTPPIKAQYVRLYPQICRRHCTLRMELLGCELSGCSEPLGMKSGHIQDYQITASSVFRTLNMDMFTWEPRKARLDKQGKVNAWT

SEQ ID NO: 19 - DNA, 1155 bp, Mus musculus
  CDS (1)..(1155)
atgaagcacttggtagcagcctggcttttggttggactcagcctcggggtgccccagttcggcaaaggtgacatttgcaacccgaacccctgtgaaaatggtggcatctgtctgtcaggactggctgatgattccttttcctgtgagtgtccagaaggcttcgcaggtccgaactgctctagtgttgtggaggttgcatcagatgaagaaaagcctacttcagcaggtccctgcatccctaacccatgccataacggaggaacctgtgagataagcgaagcctatcgaggagacacattcataggctatgtttgtaaatgtcctcggggatttaatgggattcactgtcagcacaatataaatgaatgtgaagctgagccttgcagaaatggcggaatatgtaccgaccttgttgctaactactcttgtgaatgcccaggagaatttatgggacgaaattgtcaatataaatgctctgggccattgggaatcgaaggtgggatcatatctaatcagcaaatcacagcttcatctactcaccgagctctttttggactccggaagtggtatccctactatgctcgacttaataagaagggccttataaatgcctggacagctgctgaaaatgacagatggccatggattcagataaatttgcaaagaaaaatgagagtcactggtgttattacccaaggagcaaaaaggattggaagcccagagtacataaaatcctacaaaattgcctacagcaatgacgggaagacctgggcaatgtacaaagtaaaaggcaccaatgaagagatggtctttcgtggaaatgttgataacaacacaccatatgctaattctttcacacccccaatcaaagctcagtatgtaagactctacccccaaatttgtcgaaggcattgtactttaagaatggaacttcttggctgtgagctctcaggctgttcagaacctttggggatgaaatcagggcatatacaagactaccagatcactgcctccagcgtcttcagaacactcaacatggacatgtttacttgggaaccaaggaaagccaggctggacaagcaaggcaaagtaaatgcctggacttccggccataacgaccagtcacaatggttacaggttgatcttcttgtccct

SEQ ID NO: 20 - protein, 385 aa, Mus musculus
MKHLVAAWLLVGLSLGVPQFGKGDICNPNPCENGGICLSGLADDSFSCECPEGFAGPNCSSVVEVASDEEKPTSAGPCIPNPCHNGGTCEISEAYRGDTFIGYVCKCPRGFNGIHCQHNINECEAEPCRNGGICTDLVANYSCECPGEFMGRNCQYKCSGPLGIEGGIISNQQITASSTHRALFGLRKWYPYYARLNKKGLINAWTAAENDRWPWIQINLQRKMRVTGVITQGAKRIGSPEYIKSYKIAYSNDGKTWAMYKVKGTNEEMVFRGNVDNNTPYANSFTPPIKAQYVRLYPQICRRHCTLRMELLGCELSGCSEPLGMKSGHIQDYQITASSVFRTLNMDMFTWEPRKARLDKQGKVNAWTSGHNDQSQWLQVDLLVP

SEQ ID NO: 21 - DNA, 678 bp, Mus musculus
  CDS (1)..(678)
tgtgaagctgagccttgcagaaatggcggaatatgtaccgaccttgttgctaactactcttgtgaatgcccaggagaatttatgggacgaaattgtcaatataaatgctctgggccattgggaatcgaaggtgggatcatatctaatcagcaaatcacagcttcatctactcaccgagctctttttggactccggaagtggtatccctactatgctcgacttaataagaagggccttataaatgcctggacagctgctgaaaatgacagatggccatggattcagataaatttgcaaagaaaaatgagagtcactggtgttattacccaaggagcaaaaaggattggaagcccagagtacataaaatcctacaaaattgcctacagcaatgacgggaagacctgggcaatgtacaaagtaaaaggcaccaatgaagagatggtctttcgtggaaatgttgataacaacacaccatatgctaattctttcacacccccaatcaaagctcagtatgtaagactctacccccaaatttgtcgaaggcattgtactttaagaatggaacttcttggctgtgagctctcaggctgttcagaacctttggggatgaaatcagggcatatacaagactaccagatcactgcctccagcgtcttcagaacactcaacatggacatg

SEQ ID NO: 22 - protein, 226 aa, Mus musculus
CEAEPCRNGGICTDLVANYSCECPGEFMGRNCQYKCSGPLGIEGGIISNQQITASSTHRALFGLRKWYPYYARLNKKGLINAWTAAENDRWPWIQINLQRKMRVTGVITQGAKRIGSPEYIKSYKIAYSNDGKTWAMYKVKGTNEEMVFRGNVDNNTPYANSFTPPIKAQYVRLYPQICRRHCTLRMELLGCELSGCSEPLGMKSGHIQDYQITASSVFRTLNMDM

SEQ ID NO: 23 - DNA, 285 bp, Mus musculus
  CDS (1)..(285)
tgtgaagctgagccttgcagaaatggcggaatatgtaccgaccttgttgctaactactcttgtgaatgcccaggagaatttatgggacgaaattgtcaatataaatgctctgggccattgggaatcgaaggtgggatcatatctaatcagcaaatcacagcttcatctactcaccgagctctttttggactccggaagtggtatccctactatgctcgacttaataagaagggccttataaatgcctggacagctgctgaaaatgacagatggccatggattcag

SEQ ID NO: 24 - protein, 95 aa, Mus musculus
CEAEPCRNGGICTDLVANYSCECPGEFMGRNCQYKCSGPLGIEGGIISNQQITASSTHRALFGLRKWYPYYARLNKKGLINAWTAAENDRWPWIQ

SEQ ID NO: 25 - DNA, 678 bp, Homo sapiens
  CDS (1)..(678)
tgcgaagttgagccttgcaaaaatggtggaatatgtacagatcttgttgctaactattcctgtgagtgcccaggcgaatttatgggaagaaattgtcaatacaaatgctcaggcccactgggaattgaaggtggaattatatcaaaccagcaaatcacagcttcctctactcaccgagctctttttggactccaaaaatggtatccctactatgcacgtcttaataagaaggggcttataaatgcgtggacagctgcagaaaatgacagatggccgtggattcagataaatttgcaaaggaaaatgagagttactggtgtgattacccaaggagccaagaggattggaagcccagagtatataaaatcctacaaaattgcctacagtaatgatggaaagacttgggcaatgtacaaagtgaaaggcaccaatgaagacatggtgtttcgtggaaacattgataacaacactccatatgctaactctttcacaccccccataaaagctcagtatgtaagactctatccccaagtttgtcgaagacattgcactttgcgaatggaacttcttggctgtgaactgtcgggttgttctgagcctctgggtatgaaatcaggacatatacaagactatcagatcactgcctccagcatcttcagaacgctcaacatggacatg

SEQ ID NO: 26 - protein, 226 aa, Homo sapiens
CEVEPCKNGGICTDLVANYSCECPGEFMGRNCQYKCSGPLGIEGGIISNQQITASSTHRALFGLQKWYPYYARLNKKGLINAWTAAENDRWPWIQINLQRKMRVTGVITQGAKRIGSPEYIKSYKIAYSNDGKTWAMYKVKGTNEDMVFRGNIDNNTPYANSFTPPIKAQYVRLYPQVCRRHCTLRMELLGCELSGCSEPLGMKSGHIQDYQITASSIFRTLNMDM

SEQ ID NO: 27 - DNA, 1395 bp, Mus musculus
  CDS (1)..(1395)
atggcagaatccccggctctcatcaccatcttccttttaggatatctactcagtaccgaatgtgcagttttccttgatcgtgaaaatgccaccaaaattcttacccgtccaaagagatataattcaggaaaactagaagagtttgttcgaggaaaccttgaaagagagtgtatagaagaaagatgtagttttgaagaagcacgagaagtttttgaaaacactgaaaaaactactgaattttggaagcagtatgttgatggagatcagtgtgaatcaaatccttgtttaaatggtggaatatgcaaggatgatattagttcctatgaatgctggtgccaagttggatttgaaggaaggaactgtgaattagatgcaacgtgtaacattaaaaatggcaggtgcaagcagttttgtaaaaacagtcctgataacaaggtaatttgttcctgcactgagggataccaacttgcagaagaccagaagtcctgtgaaccaacagttccatttccatgtgggagagcttctatttcatacagttctaaaaagatcacgagagctgagactgttttctctaatatggactatgaaaattctactgaagctgtattcattcaagatgacatcactgatggtgccattcttaataacgtcactgaaagtagtgaatcacttaatgacttcactcgagttgttggtggagaaaacgcaaaaccgggtcaaatcccttggcaggtcattttaaatggtgaaattgaggcattctgtggaggtgccatcattaatgaaaaatggattgtaactgctgcccactgtcttaaacctggtgataaaattgaggttgttgctggtgaatataacattgataagaaggaagacacagaacaaaggagaaatgtgattcgaactatccctcatcaccagtacaatgcaactattaataagtatagtcatgacattgccttgctggaactggataaacctttaatactaaacagctatgtaacacctatctgtgttgccaatagggaatatacaaatatcttcctcaagtttggttctggctatgtcagtggctggggaaaagtcttcaacaaagggagacaggcttccattcttcagtaccttagagttccactggtggatagagccacatgccttaggtccacaacattcactatctataacaacatgttctgtgcaggctaccgtgaaggaggcaaagattcgtgtgaaggagatagtgggggaccccatgttactgaagtagaagggacaagtttcttaactggcattattagctggggtgaagaatgtgcaatgaaaggcaaatatggaatatatactaaggtttcccggtacgtcaactggattaaggaaaaaacaaagctaacttaa

SEQ ID NO: 28 - protein, 464 aa, Mus musculus
MAESPALITIFLLGYLLSTECAVFLDRENATKILTRPKRYNSGKLEEFVRGNLERECIEERCSFEEAREVFENTEKTTEFWKQYVDGDQCESNPCLNGGICKDDISSYECWCQVGFEGRNCELDATCNIKNGRCKQFCKNSPDNKVICSCTEGYQLAEDQKSCEPTVPFPCGRASISYSSKKITRAETVFSNMDYENSTEAVFIQDDITDGAILNNVTESSESLNDFTRVVGGENAKPGQIPWQVILNGEIEAFCGGAIINEKWIVTAAHCLKPGDKIEVVAGEYNIDKKEDTEQRRNVIRTIPHHQYNATINKYSHDIALLELDKPLILNSYVTPICVANREYTNIFLKFGSGYVSGWGKVFNKGRQASILQYLRVPLVDRATCLRSTTFTIYNNMFCAGYREGGKDSCEGDSGGPHVTEVEGTSFLTGIISWGEECAMKGKYGIYTKVSRYVNWIKEKTKLT

SEQ ID NO: 29 - DNA, 102 bp, Mus musculus
  CDS (1)..(102)
tgtgaatcaaatccttgtttaaatggtggaatatgcaaggatgatattagttcctatgaatgctggtgccaagttggatttgaaggaaggaactgtgaatta

SEQ ID NO: 30 - protein, 34 aa, Mus musculus
CESNPCLNGGICKDDISSYECWCQVGFEGRNCEL

SEQ ID NO: 31 - protein, 12 aa, Mus musculus
CTDLVANYSCEC

SEQ ID NO: 32 - DNA, 27 bp, Artificial Sequence
  synthetic DNA
tgcaacccgaacccctgtgaaaatggt

SEQ ID NO: 33 - DNA, 25 bp, Artificial Sequence
  synthetic DNA
ttcctcctctgcgcagcccagcagc

SEQ ID NO: 34 - DNA, 26 bp, Artificial Sequence
  synthetic DNA
tgtgaagctgagccttgcagaaatgg

SEQ ID NO: 35 - DNA, 26 bp, Artificial Sequence
  synthetic DNA
atattgacaatttcgtcccataaatt

SEQ ID NO: 36 - DNA, 34 bp, Artificial Sequence
  synthetic DNA
aaagatcttgtgaatcaaatccttgtttaaatgg

SEQ ID NO: 37 - DNA, 35 bp, Artificial Sequence
  synthetic DNA
ttctcgagttaattcacagttccttccttcaaatc

(19) United States
(12) Patent Application Publication
(10) Pub. No.: US 2010/0087005 A1
(43) Pub. Date: Apr. 8, 2010

(54) AUXILIARY REAGENT FOR GENE TRANSFER

(76) Inventors: Chiaki Hidai, Tokyo (JP); Hisataka Kitano, Tokyo (JP)

Correspondence Address: BIRCH STEWART KOLASCH & BIRCH, PO BOX 747, FALLS CHURCH, VA, 22040-0747, US

(21) Appl. No.: 12/444,966

(22) PCT Filed: Oct. 10, 2007
(86) PCT No.: PCT/JP2007/070221

§ 371 Date: Apr. 9, 2009

(30) Foreign Application Priority Data
Oct. 10, 2006 (JP) 2006-276322

(51) Int. Cl. C12N 15/63; C07K 7/08; C07H 21/04; C12N 1/21; C12N 15/74; C12N 5/10

(52) U.S. Cl. 435/455; 530/327; 536/23.1; 435/320.1; 435/252.3; 435/471; 435/325

(57) ABSTRACT

The present invention provides a means for introducing a gene of interest into a host cell with a higher degree of efficiency regardless of the gene transfer method employed and the type of the host cell. The auxiliary reagent for gene transfer of the present invention is characterized in that it comprises a peptide comprising a specific amino acid sequence represented by formula (I), a mutant or derivative thereof, or a salt of the peptide, the mutant or the derivative.

### TECHNICAL FIELD

The present invention relates to an auxiliary reagent for gene transfer which increases the efficiency of gene transfer into a host cell.

### BACKGROUND ART

Methods for introducing a gene of interest into a cultured cell to observe change of the cell are used, for example, when analyzing the function of a gene. Such gene transfer methods have become significantly popular because they can be more easily carried out and are more inexpensive compared to methods for preparing a transgenic animal.

Many methods for gene transfer into a cultured cell are known. Examples thereof include an electroporation method, a liposome method (lipoplex method), a polyplex method, a peptide method and a virus vector method. In particular, a liposome method, polyplex method, etc., in which a gene is introduced into a host cell using a reagent which has affinity to both the gene to be introduced and the host cell, are often used because of efficiency and easiness (for example, see: Gao X et al., Cationic liposome mediated gene transfer., Gene Ther., vol. 2, pp. 710-722, 1995; and J Rejman et al., Role of clathrin and caveolae mediated endocytosis in gene transfer mediated by lipo and polyplex., Molecular therapy, vol. 12, pp. 468-474, 2005).

However, when employing a conventional gene transfer method, the transfer efficiency often varies depending on the type of host cell. Further, some reagents have cytotoxicity. Therefore, when using such a reagent, it cannot be added in a sufficient amount, and as a result, it is difficult to efficiently perform gene transfer. For this reason, the progress of experiments and researches is often hindered.

### DISCLOSURE OF THE INVENTION

The problem to be solved by the present invention is to provide a means for introducing a gene of interest into a host cell with a higher degree of efficiency regardless of the gene transfer method employed and the type of the host cell, and to provide a gene transfer method using the means and a kit for gene transfer.

The present inventor diligently made researches in order to solve the above-described problem and found that the above-described problem can be solved by using a specific peptide as an auxiliary reagent when introducing a gene of interest into a host cell, and thus the present invention was achieved.

More specifically, the present invention is as follows:

(1) An auxiliary reagent for gene transfer comprising a peptide of (a) or (b) below, a derivative thereof, or a salt of the peptide or the derivative:
(a) a peptide comprising an amino acid sequence represented by the following formula (I):

C-X-[D/N]-X-X-X-X-[F/Y]-X-C-X-C (I)

wherein X represents any amino acid residue, C represents cysteine, [D/N] represents aspartic acid or asparagine, and [F/Y] represents phenylalanine or tyrosine;
(b) a peptide, which comprises the amino acid sequence represented by the formula (I) having deletion, substitution or addition of one or several amino acids, and which has activity to increase the efficiency of gene transfer into a host cell.
(2) A DNA encoding a peptide of (a) or (b) below:
(a) a peptide comprising an amino acid sequence represented by the following formula (I):

C-X-[D/N]-X-X-X-X-[F/Y]-X-C-X-C (I)

wherein X represents any amino acid residue, C represents cysteine, [D/N] represents aspartic acid or asparagine, and [F/Y] represents phenylalanine or tyrosine;
(b) a peptide, which comprises the amino acid sequence represented by the formula (I) having deletion, substitution or addition of one or several amino acids, and which has activity to increase the efficiency of gene transfer into a host cell.
(3) A recombinant vector comprising the DNA according to item (2).
(4) A transformant comprising the recombinant vector according to item (3).
(5) A gene transfer method, wherein a gene is introduced into a host cell in the presence of the reagent according to item (1).
(6) A kit for gene transfer, comprising the reagent according to item (1).

### BRIEF DESCRIPTION OF THE DRAWINGS

FIG. 1 is a graph showing comparison of the activity to increase gene transfer efficiency in transfer of gene into vascular endothelial cell (Pro5) using jet-PEI based on the presence/absence of Del-1 protein.

FIG. 2 is a graph showing comparison of the activity to increase gene transfer efficiency in transfer of gene into vascular endothelial cell (Pro5) using Lipofectamine-2000 based on the presence/absence of Del-1 protein.

FIG. 3 is a graph showing comparison of the activity to increase gene transfer efficiency in transfer of gene into renal epithelium-derived cell (Cos1) based on the presence/absence of Del-1 protein.

FIG. 4 is a graph showing comparison of the activity to increase gene transfer efficiency among different proteins.

FIG. 5 is a graph showing alkaline phosphatase activity in serum of wild type mouse (WT) and transgenic mouse (TG).

### BEST MODE FOR CARRYING OUT THE INVENTION

Hereinafter, the present invention will be described in detail. The scope of the present invention is not limited to the description. In addition to the following examples, the present invention can be suitably changed and then practiced within a range in which the effects of the present invention are not reduced.

Note that the entire specification of Japanese Patent Application No. 2006-276322, to which priority is claimed by the present application, is incorporated herein. In addition, all the publications such as prior art documents, laid-open publications, patents and other patent documents cited herein are incorporated herein by reference.

### 1. Auxiliary Reagent for Gene Transfer

As described above, the auxiliary reagent for gene transfer of the present invention (hereinafter referred to as the auxiliary reagent of the present invention) comprises a peptide of the following (a):

(a) a peptide comprising an amino acid sequence represented by the following formula (I):

| | | |
|---|---|---|
| C-X-[D/N]-X-X-X-X-[F/Y]-X-C-X-C | (SEQ ID NO: 1) | (I) |

In the above-described peptide in (a), the amino acid sequence of formula (I) is described using one-character codes. For example, C, D, N, F and Y mean cysteine (Cys), aspartic acid (Asp), asparagine (Asn), phenylalanine (Phe) and tyrosine (Tyr), respectively. [D/N] represents aspartic acid or asparagine, and [F/Y] represents phenylalanine or tyrosine. X represents any amino acid residue, which can be generally selected from 20 types of amino acid residues, i.e., A (Ala), R (Arg), D (Asp), N (Asn), C (Cys), Q (Gln), E (Glu), G (Gly), H (His), I (Ile), L (Leu), K (Lys), M (Met), F (Phe), P (Pro), S (Ser), T (Thr), W (Trp), Y (Tyr) and V (Val). In the present specification, other amino acid sequences may also be described using one-character codes like the case of formula (I).

The aforementioned amino acid sequence of formula (I) includes the following amino acid sequences represented by SEQ ID NOS: 2 to 5. Among them, the amino acid sequence represented by SEQ ID NO: 2 is preferred.

| | |
|---|---|
| C-X-D-X-X-X-X-Y-X-C-X-C | (SEQ ID NO: 2) |
| C-X-D-X-X-X-X-F-X-C-X-C | (SEQ ID NO: 3) |
| C-X-N-X-X-X-X-Y-X-C-X-C | (SEQ ID NO: 4) |
| C-X-N-X-X-X-X-F-X-C-X-C | (SEQ ID NO: 5) |

Specifically, preferred examples of the amino acid sequence of formula (I) are the following amino acid sequences represented by SEQ ID NOS: 6 and 7, and the amino acid sequence represented by SEQ ID NO: 6 is more preferred.

| | |
|---|---|
| C-T-[D/N]-L-V-A-N-[F/Y]-S-C-E-C | (SEQ ID NO: 6) |
| C-K-[D/N]-D-I-S-S-[F/Y]-E-C-W-C | (SEQ ID NO: 7) |

As used herein, “peptide” refers to a structure in which at least 2 amino acids are bound together by peptide bond. Examples thereof include oligopeptide and polypeptide. A substance in which a given three-dimensional structure is formed by polypeptides is called “protein”. In the present invention, protein of this kind is also included in the above-described “peptide”. Therefore, the peptide included in the auxiliary reagent of the present invention may be any of oligopeptide, polypeptide and protein.

As described above, the auxiliary reagent of the present invention may comprise a peptide of the following (b):

(b) a peptide, which comprises the amino acid sequence represented by the formula (I) having deletion, substitution or addition of one or several amino acids, and which has activity to increase the efficiency of gene transfer into a host cell.

In this regard, examples of the above-described “amino acid sequence having deletion, substitution or addition of one or several amino acids” include, but are not limited to, amino acid sequences having deletion, substitution or addition of about 1 to 5 amino acids, and preferably about 1 to 2 amino acids. In the present invention, the amino acid sequence represented by the formula (I) without having deletion, substitution or addition of amino acid residues at positions 1, 3, 8, 10 and 12 (C, [D/N], [F/Y], C and C in this order), and/or the amino acid sequence represented by the formula (I) without having deletion or addition of amino acid residues at positions 2, 4-7, 9 and 11 (all of them are X) are preferred. The introduction of mutation such as deletion, substitution and addition can be carried out using a kit for introducing mutation utilizing site-directed mutagenesis such as GeneTailor™ Site-Directed Mutagenesis System (manufactured by Invitrogen Corporation) and TaKaRa Site-Directed Mutagenesis System (Mutan-K, Mutan-Super Express K, etc.: manufactured by Takara Bio Inc.). Further, whether or not a peptide has the above-described introduced mutation (deletion, substitution or addition) can be confirmed using various amino acid sequence determination methods, structural analysis methods using X-ray, NMR and the like, etc.

In the present invention, activity to increase the efficiency of gene transfer means enhancement of endocytosis and phagocytosis of the host cell. The activity can be measured, for example, by means of reporter assay using a reporter gene such as β-galactosidase, luciferase gene, chloramphenicol acetyltransferase (CAT) gene and GFP or mutant thereof (EGFP, YFP, BFP, etc.) as a gene to be introduced.

As long as the aforementioned peptide of (a) or (b) included in the auxiliary reagent of the present invention is a peptide comprising the amino acid sequence represented by the formula (I) or a peptide comprising the amino acid sequence having the aforementioned deletion, substitution or addition, the number of residues of constituent amino acids of the peptide is not limited. 12 or more residues are preferred. For example, the peptide may have 12 to 100 residues, 12 to 50 residues, or 12 to 30 residues.

The aforementioned peptide of (a) or (b) may be a peptide from a natural product or a peptide obtained by chemical synthesis. There is no limitation regarding this, but peptides from natural products are preferred since many of such peptides do not exert adverse effects such as cytotoxicity.

Examples of peptides from natural products include naturally-occurring oligopeptides, polypeptides and proteins, and fragments thereof. The peptide from a natural product can be directly obtained from the natural product using a publicly-known collection method and purification method. Alternatively, a gene encoding the peptide is incorporated into any of various expression vectors or the like and introduced into a cell by means of a publicly-known genetic engineering technique, and after expressed, the peptide may be obtained by means of a publicly-known collection method and purification method. Alternatively, the peptide is produced by the cell-free protein synthesizing system using a commercially-available kit such as reagent kits PROTEIOS™ (Toyobo Co., Ltd.) and TNT™ System (Promega), a synthesis device PG-Mate™ (Toyobo Co., Ltd.), RTS (Roche Diagnostics K.K.), etc., and then the peptide may be obtained by means of a publicly-known collection method and purification method. There is no limitation regarding this.

The chemically-synthesized peptide can be obtained using a publicly-known peptide synthesis method. Examples of the synthesis methods include azide method, acid chloride method, acid anhydride method, mixed acid anhydride method, DCC method, active ester method, carboimidazole method and oxidation-reduction method. To such synthesis, solid-phase synthesis or liquid-phase synthesis may be applied. A commercially-available peptide synthesis apparatus may be used. After synthetic reaction, the peptide can be purified using a combination of publicly-known purification methods such as chromatography.

Hereinafter, as examples of peptides, which are the aforementioned peptide of (a) or (b), and which correspond to a peptide from a natural product, (i) developmentally regulated endothelial cell locus-1 (Del-1: developmentally endothelial locus-1) protein and a partial fragment thereof and (ii) blood coagulation factor IX will be described.

Del-1 protein is an extracellular matrix protein having epidermal growth factor (EGF)-like domains and discoidin I-like domains, which consists of the amino acid sequence represented by SEQ ID NO: 10. It is known that EGF1, EGF2 and EGF3 exist as EGF-like domains of Del-1 protein, and that Discoidin1 and Discoidin2 exist as discoidin I-like domains. Among the above-described various domains, the present inventor focused their attention on EGF3, and further focused their attention on a region consisting of specific 12 amino acid residues in the amino acid sequence constituting EGF3 (SEQ ID NO: 12) (C-T-D-L-V-A-N-Y-S-C-E-C (SEQ ID NO: 8)). Further, the present inventor found that peptides including the specific region have activity to increase the efficiency of gene transfer. That is, examples of peptides, which are the aforementioned peptide of (a) or (b), and which correspond to a peptide from a natural product, include full-length Del-1 protein, partial fragment of Del-1 protein consisting of EGF3, and various partial fragments of Del-1 protein including EGF3 (see Table 1). Regarding the details of Del-1 protein and partial fragments thereof, the entire description in International Publication WO2005/0001093 pamphlet can be referred to. Partial fragments other than those having any of the amino acid sequences shown in Table 1 can be included in the present invention. For example, a partial fragment having an amino acid sequence in which a signal peptide portion is deleted from “full-length Del-1” in Table 1 can also be included in the present invention. The above-described “amino acid sequence in which a signal peptide portion is deleted from full-length Del-1” is an amino acid sequence encoded by a nucleotide sequence consisting of bases at positions 694-2061 in the nucleotide sequence represented by SEQ ID NO: 9, i.e., an amino acid sequence consisting of amino acid residues at positions 26-480 in the amino acid sequence represented by SEQ ID NO: 10.

TABLE 1

| Name of | | | |
|---|---|---|---|
| fragment or the like | Region * | Type | SEQ ID NO: |
| Full-length Del-1 | 619-2061 | DNA | 9 |
| | 1-480 | Protein | 10 |
| EGF3 | 985-1089 | DNA | 11 |
| | 123-157 | Protein | 12 |
| 4-1 | 619-1662 | DNA | 13 |
| | 1-348 | Protein | 14 |
| 4-15 | 619-1713 | DNA | 15 |
| | 1-365 | Protein | 16 |
| 4-14 | 619-1722 | DNA | 17 |
| | 1-368 | Protein | 18 |
| 4-13 | 619-1773 | DNA | 19 |
| | 1-385 | Protein | 20 |
| XY | 985-1662 | DNA | 21 |
| | 123-348 | Protein | 22 |
| XC | 985-1269 | DNA | 23 |
| | 123-217 | Protein | 24 |
| human XY | — | DNA | 25 |
| | — | Protein | 26 |

* Regions in DNAs are indicated by base numbers, and regions in proteins are indicated by amino acid numbers. Note that base numbers and amino acid numbers used herein are corresponding numbers on the full-length Del-1 sequence. That is, in the case of base numbers, they are base numbers on the nucleotide sequence represented by SEQ ID NO: 9, and in the case of amino acid sequences, numbers of amino acid residues on the amino acid sequence represented by SEQ ID NO: 10 are used.

Blood coagulation factor IX is a protein consisting of the amino acid sequence represented by SEQ ID NO: 28, and it is a single-stranded glycoprotein having Gla domain, EGF domain and protease domain in this order from the N-terminus. In this regard, the EGF domain of the factor IX is constituted by a sequence (SEQ ID NO: 30) consisting of amino acids at positions 90-123 in the amino acid sequence represented by SEQ ID NO: 28. The present inventor focused their attention on the fact that a region consisting of specific 12 amino acid residues (C-K-D-D-1-S-S-Y-E-C-W-C (SEQ ID NO: 31)) is included in the amino acid sequence constituting the EGF domain of the factor IX as in the case of the aforementioned EGF3 of Del-1 protein (EGF-like domain), and found that peptides including this region can increase the efficiency of gene transfer. That is, examples of peptides, which are the aforementioned peptide of (a) or (b), and which correspond to a peptide from a natural product, include full-length factor IX, peptide fragment consisting of the amino acid sequence represented by SEQ ID NO: 31, various partial fragments including the fragment, partial fragment consisting of the EGF domain, and various partial fragments including the EGF domain. The nucleotide sequence of DNA encoding the full-length factor IX is represented by SEQ ID NO: 27, and the nucleotide sequence of DNA encoding the EGF domain of the factor IX is represented by SEQ ID NO: 29.

The auxiliary reagent of the present invention may include a derivative of the aforementioned peptide of (a) or (b) together with or instead of the peptide. The “derivative” means that all derivatives which can be prepared from the aforementioned peptide of (a) or (b) are included. Examples thereof include a derivative in which a part of constituent amino acids of the peptide are replaced by normatural amino acids and a derivative in which a part of constituent amino acids of the peptide (mainly side chain thereof) are chemically modified.

The auxiliary reagent of the present invention may include a salt of the aforementioned peptide of (a) or (b) and/or a salt of a derivative of the peptide together with or instead of the peptide and/or the derivative. These salts are preferably physiologically-acceptable acid addition salts or basic salts. Examples of acid addition salts include salts using inorganic acids such as hydrochloric acid, phosphoric acid, hydrobromic acid and sulfuric acid, and salts using organic acids such as acetic acid, formic acid, propionic acid, fumaric acid, maleic acid, succinic acid, tartaric acid, citric acid, malic acid, oxalic acid, benzoic acid, methanesulfonic acid and benzenesulfonic acid. Examples of basic salts include salts using inorganic bases such as sodium hydroxide, potassium hydroxide, ammonium hydroxide and magnesium hydroxide, and salts using organic bases such as caffeine, piperidine, trimethylamine and pyridine.

Salts can be prepared using a suitable acid such as hydrochloric acid or a suitable base such as sodium hydroxide. For example, salts can be prepared by treatment in water or a solution containing an inactive water-miscible organic solvent such as methanol, ethanol and dioxane using a standard protocol.

The auxiliary reagent of the present invention may consist of the above-described peptide of (a) or (b), a derivative thereof, or a salt of the peptide or the derivative, or may include: the peptide, a derivative thereof, or a salt of the peptide or the derivative; and other components. There is no limitation regarding this. Examples of other components include buffer solutions such as PBS and Tris-HCl, and additives such as sodium azide and glycerol. When the auxiliary reagent of the present invention includes other components, each of the contents thereof can be suitably determined within a range in which the activity to increase the efficiency of gene transfer provided by the above-described peptide, a derivative thereof, or a salt of the peptide or the derivative is not significantly blocked.

### 2. DNA

The DNA of the present invention includes DNAs consisting of a nucleotide sequence encoding an amino acid sequence constituting the aforementioned peptide of (a) or (b). Therefore, the DNA of the present invention may consist of only a DNA consisting of a nucleotide sequence encoding the aforementioned peptide of (a) or (b), or may include the DNA and another publicly-known nucleotide sequence required for gene expression (transcription promoter, SD sequence, Kozak sequence, terminator, etc.). There is no limitation regarding this. In the nucleotide sequence encoding the aforementioned peptide of (a) or (b), the type of codon is not limited. For example, after transcription, a codon which is generally used in mammals such as human may be used, or a codon which is generally used in microorganisms such as E. coli and yeast, plants or the like may be used. This can be suitably selected or designed.

In addition, in the present invention, a DNA, which can hybridize to a DNA consisting of a nucleotide sequence complementary to a DNA including a nucleotide sequence encoding the aforementioned peptide of (a) or (b) under stringent conditions, and which encodes a protein having activity to increase the efficiency of gene transfer, can also be included in the DNA of the present invention. In this regard, the term “stringent conditions” refers to, for example, conditions in which the concentration of salt (sodium) is 150 to 900 mM and the temperature is 55 to 75° C., and preferably conditions in which the concentration of salt (sodium) is 150 to 200 mM and the temperature is 60 to 70° C.

### 3. Recombinant Vector and Transformant

### (1) Recombinant Vector Including DNA

The recombinant vector of the present invention can be obtained by linking (inserting) the DNA of the present invention to (into) a suitable vector. A vector into which the DNA of the present invention is to be inserted is not particularly limited as long as it can be replicated in a host. Examples thereof include plasmid DNA, phage DNA, virus, etc.

Examples of plasmid DNAs include E. coli-derived plasmid, Bacillus subtilis-derived plasmid and yeast-derived plasmid. Examples of phage DNAs include X, phage. Examples of viruses include adenovirus and retrovirus.

Other than promoters and the DNA of the present invention, if desired, cis elements such as enhancers, splicing signals, poly(A) addition signals, selection markers, ribosome binding sequences (SD sequences) or the like may be linked to the vector of the present invention. Examples of selection markers include dihydrofolate reductase gene, ampicillin resistance gene and neomycin resistance gene.

### (2) Transformant

The transformant of the present invention can be obtained by introducing the recombinant vector of the present invention into a host so that a gene of interest can be expressed. The host is not limited as long as it can express the DNA of the present invention. Examples of hosts which may be used in the invention include bacteria, yeasts and animal cells well-known in the art. Alternatively, experimental animals such as mouse may be used.

When a bacterium is used as a host, the recombinant vector of the invention is capable of autonomous replication in the bacterium, and at the same time, may also comprise a promoter, a ribosome binding sequence, the DNA of the invention and a transcription termination sequence. Specific examples of bacteria which may be used in the invention include E. coli (Escherichia coli). Examples of promoters which may be used in the invention include lac promoter. Examples of methods for introducing a vector into a bacterium include calcium ion method.

When an yeast is used as a host, Saccharomyces cerevisiae or the like may be used. A promoter which may be used in this case is not particularly limited as long as it allows expression in yeast. Examples thereof include gall promoter. Examples of methods for introducing a vector into an yeast include an electroporation method and spheroplast method.

When an animal cell is used as a host, CHO cells, rat GH3 cells, human HEK293 cells or the like may be used. Examples of promoters which may be used in the invention include SV40 promoter. Examples of methods for introducing a vector into an animal cell include an electroporation method and calcium phosphate method.

Further, for gene transfer into an animal or plant, a virus vector method, lipofection method or the like may be used. It is also possible to introduce a gene into a germ cell or ES cell to create a genetically modified animal.

### 4. Gene Transfer Method

As described above, the gene transfer method of the present invention is characterized in that a gene is introduced into a host cell in the presence of the auxiliary reagent of the present invention.

Specifically, the method of the present invention is characterized in that, in a reaction of gene transfer into a host cell using any of various publicly-known gene transfer methods such as a liposome method (lipoplex method), a polyplex method, a peptide method, an electroporation method and a virus vector method, the auxiliary reagent of the present invention is caused to exist in the same reaction system at least at the time of gene transfer. Therefore, as long as the auxiliary reagent of the present invention is caused to exist as described above, it may be added to the reaction system together with various gene transfer reagents and/or a gene of interest to be introduced, or these substances may be added separately. There is no limitation regarding this.

As long as the auxiliary reagent of the present invention is used in a manner as described above, the method of the present invention include any step. There is no limitation regarding this, but in general, steps according to protocols of various publicly-known gene introduction methods are preferably included.

The method of the present invention using the auxiliary reagent of the present invention can increase the efficiency of gene transfer into a host cell more effectively compared to the cases where various gene transfer methods are carried out according to general protocols. In addition, the method of the present invention can increase the efficiency of gene transfer regardless of the type of host cell to be used.

### 5. Kit for Gene Transfer

As described above, the kit for gene transfer of the present invention is characterized in that it includes the auxiliary reagent of the present invention as a constituent.

When a gene is introduced into various host cells using various publicly-known gene transfer methods, the kit of the present invention can further increase the transfer efficiency. Therefore, the kit of the present invention is significantly useful in the fields of experiments, researches, etc.

The kit of the present invention may include constituents other than the auxiliary reagent of the present invention. Examples of other constituents include, but are not limited to, buffer solutions such as PBS and Tris-HCl, additives such as sodium azide and glycerol, an experiment manual, a gene transfer reagent, etc.

### EXAMPLES

Hereinafter, the present invention will be specifically described by way of illustrative examples, but the present invention is not limited thereto.

### Example 1

### Increase of Transfer Efficiency in Gene Transfer Method Using Polyplex Method

Experiment of transfer of lacZ gene into a Pro5 cell (vascular endothelial cell) was carried out using a gene transfer reagent jet-PEI (Polyplus Transfection) in which linear polyethyleneimine (PEI), which is a cationic water-soluble polymer, is based.

Firstly, using a pAPtag-4 vector (GenHunter), an expression vector, which includes a DNA encoding a fusion protein of alkaline phosphatase and Del-1 (excluding the signal peptide portion), was constructed as follows. The pAPtag-4 vector as used herein can transiently express a protein in which a signal peptide and alkaline phosphatase are fused to the N-terminus of a protein of interest by inserting a DNA encoding the protein of interest into a predetermined position.

An RNA was extracted from a mouse embryo on day 9 to day 12 after fertilization using TRIzol (Invitrogen Corporation). Using this RNA as a template, a reverse transcription reaction was performed according to the ordinary method utilizing Superscript II (Invitrogen Corporation) to prepare a cDNA.

Using this cDNA as a template, a nucleotide sequence encoding an amino acid sequence in which the signal peptide portion is deleted from the amino acid sequence of the full-length Del-1 represented by SEQ ID NO: 10, i.e., a nucleotide sequence consisting of bases at positions 694 to 2061 in the nucleotide sequence represented by SEQ ID NO: 9, was amplified by means of PCR. In order to enable insertion of the amplified product into the pAPtag-4 vector, F and R primers, in which a restriction enzyme recognition sequence was introduced to the 5′ end, were synthesized.

F primer:

| | |
|---|---|
| 5′-tgcaacccgaacccctgtgaaaatggt-3′ | (SEQ ID NO: 32) |

R primer:

| | |
|---|---|
| 5′-ttcctcctctgcgcagcccagcagc-3′ | (SEQ ID NO: 33) |

The above-described PCR was performed using a reaction solution with the following composition under the following reaction conditions.

### <Composition of Reaction Solution>

| | |
|---|---|
| Template DNA (50 ng/μL) | 5 μL |
| (cDNA produced by a reverse | |
| transcriptase): | |
| F primer (50 pM): | 1 μL |
| R primer (50 pM): | 1 μL |
| dNTP (2.5 mM for each): | 4 μL |
| Taq polymerase (5 unit/μL): | 1 μL |
| Buffer: | 5 μL |
| (20 mM Tris-HCl (pH 8.3), | |
| 100 mM KCl, 20 mM MgCl₂) | |
| Sterile water: | Appropriate amount (about 33 μL) |
| Total: | 50 μL |

### <Reaction Conditions>

In one cycle, reaction conditions are as follows: “at 94° C. for 30 seconds (heat denaturation/disassociation)→at 62° C. for 30 seconds (annealing)→at 72° C. for 90 seconds (synthesis/extension)”. In total, 35 cycles were carried out.

After performing PCR, both the amplified fragment and the pAPtag-4 vector were treated with Bgl II and Xho I. After that, the amplified fragment was inserted into the pAPtag-4 vector using the publicly-known genetic engineering technique to construct an expression vector which can express a fusion protein of interest.

Next, the expression vector was introduced into a Cos1 cell using the ordinary method to prepare a conditioned medium. As a negative control (Dell(−)), only the pAPtag-4 vector was used. Next, according to the instructions attached to jet-PEI, a Pro5 cell and cDNA of a lacZ gene were prepared. To a culture solution at the time of introduction of lacZ gene, a fusion protein of alkaline phosphatase and Del-1 was added (Del-1 (+)), and to the negative control, alkaline phosphatase in an amount to have alkaline phosphatase activity equivalent to that of the above-described added fusion protein was added. 72 hours after the introduction of lacZ gene, the Pro5 cell was collected to measure β-galactosidase activity.

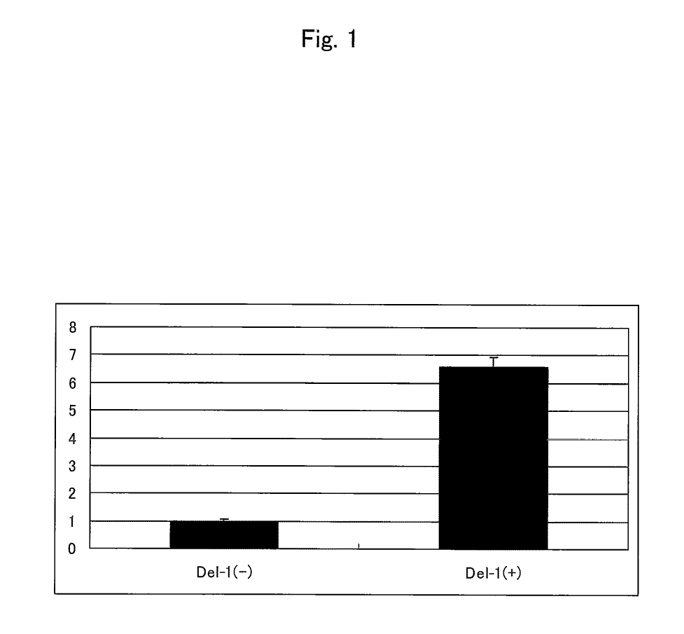

Results of the experiment are shown in FIG. 1.

Because of the addition of the fusion protein of Del-1, the efficiency of gene transfer was successfully increased by about 6.5 times.

### Example 2

### Increase of Transfer Efficiency in Gene Transfer Method Using Liposome Method

Experiment of transfer of lacZ gene into a Pro5 cell (vascular endothelial cell) was carried out using a gene transfer reagent Lipofectamine 2000 (Invitrogen Corporation) in which cationic lipid is based.

Firstly, in a manner similar to that in Example 1, using a pAPtag-4 vector (GenHunter), an expression vector, which includes a DNA encoding a fusion protein of alkaline phosphatase and Del-1 (excluding the signal peptide portion), was constructed, and the expression vector was introduced into a Cos1 cell to prepare a conditioned medium. As a negative control, only the pAPtag-4 vector was used. Next, according to the instructions attached to Lipofectamine 2000, a Pro5 cell and cDNA of a lacZ gene were prepared. To a culture solution at the time of introduction of lacZ gene, a fusion protein of alkaline phosphatase and Del-1 was added (Del-1 (+)), and to the negative control (Del-1(−)), alkaline phosphatase in an amount to have alkaline phosphatase activity equivalent to that of the above-described added fusion protein was added. 72 hours after the introduction of lacZ gene, the Pro5 cell was collected to measure β-galactosidase activity.

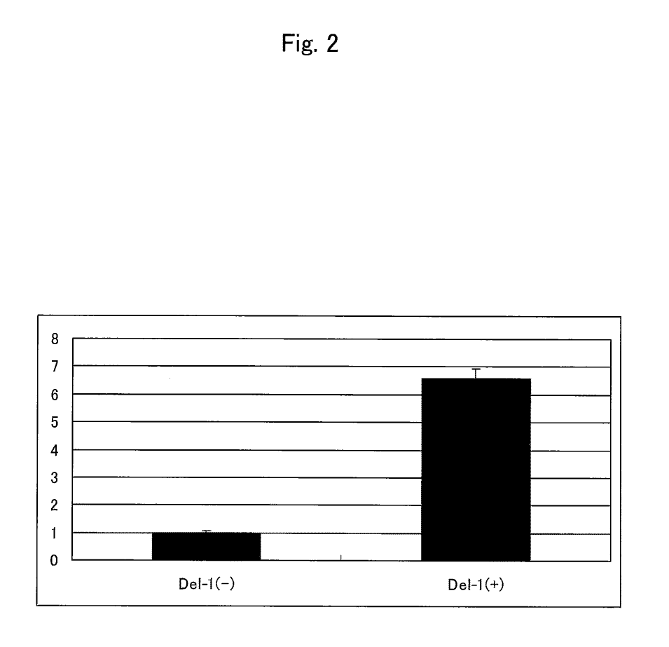

Results of the experiment are shown in FIG. 2.

Because of the addition of the fusion protein of Del-1, the efficiency of gene transfer was successfully increased by about 15 times.

### Example 3

### Increase of Gene Transfer Efficiency with Respect to Renal Epithelium-Derived Cell (Cos1)

Experiment of transfer of lacZ gene into a Cos1 cell was carried out using jet-PEI (Polyplus Transfection).

Firstly, in a manner similar to that in Example 1, using a pAPtag-4 vector (GenHunter), an expression vector, which includes a DNA encoding a fusion protein of alkaline phosphatase and Del-1 (excluding the signal peptide portion), was constructed, and the expression vector was introduced into a Cos1 cell to prepare a conditioned medium. As a negative control (Del-1 (−)), only the pAPtag-4 vector was used. Next, according to the instructions attached to jet-PEI, a Cos1 cell and cDNA of a lacZ gene were prepared. To a culture solution at the time of introduction of lacZ gene, a fusion protein of alkaline phosphatase and Del-1 was added (Del-1 (+)), and to the negative control, alkaline phosphatase in an amount to have alkaline phosphatase activity equivalent to that of the above-described added fusion protein was added. 72 hours after the introduction of lacZ gene, the Pro5 cell was collected to measure β-galactosidase activity.

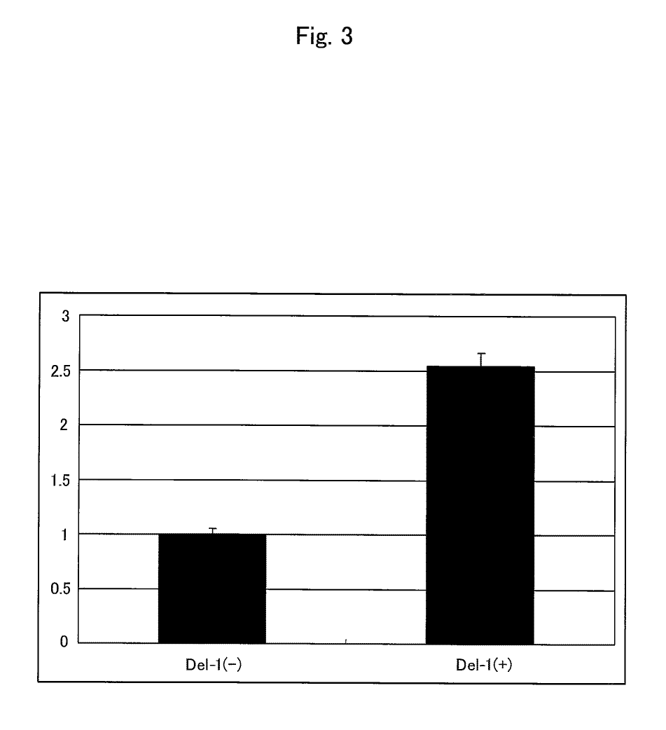

Results of the experiment are shown in FIG. 3.

With respect to the effect of increasing the gene transfer efficiency by addition of the fusion protein of Del-1, when using the Pro5 cell as a host cell (Example 1), the efficiency was increased by about 6.5 times, and when using the Cos1 cell in this example, the efficiency was increased by about 2.5 times. The apparent effect was small, but a system in which Cos1 cell is used as a host cell is a system by which high-efficiency gene transfer can be realized from the start. Therefore, since the high efficiency of introduction in a system using Cos1 cell was successfully increased further by about 2.5 times, it can be said that a significantly beneficial effect is exerted.

### Example 4

### Examination Regarding Protein Having Activity to Increase the Efficiency of Gene Transfer

Examination was made as to what kind of amino acid sequence in Del-1 has activity to increase the efficiency of gene transfer and whether or not there is an amino acid sequence of a protein other than Del-1 having the activity.

Specifically, each of the following 4 types of proteins (i) to (iv) was subjected to examination for comparison of activity to increase the gene transfer efficiency.

(i) Del-1 (excluding the signal peptide portion)
(ii) EGF3 domain of Del-1 (EGF 3rd of Del-1; SEQ ID NO: 11)
(iii) protein in which the amino acid at position 136 of EGF3 domain of Del-1, aspartic acid is replaced by glutamic acid (EGF 3rd of Del-1(D136E))
(iv) EGF domain of blood coagulation factor IX (F-IX)

Firstly, in a manner similar to that in Example 1, using a pAPtag-4 vector (GenHunter), expression vectors, each of which includes a DNA encoding a fusion protein of alkaline phosphatase and each of the above-described proteins, were constructed.

As DNAs encoding (ii) EGF 3rd of Del-1 or (iv) F-IX, products obtained by performing PCR similar to that in Example 1 were used except that the following primers were used, respectively.

Primers for obtaining DNA encoding (ii) EGF 3rd of Del-1
F primer:

| | |
|---|---|
| 5′-tgtgaagctgagccttgcagaaatgg-3′ | (SEQ ID NO: 34) |

R primer:

| | |
|---|---|
| 5′-atattgacaatttcgtcccataaatt-3′ | (SEQ ID NO: 35) |

Primers for obtaining DNA encoding (iv) F-IX
F primer:

(SEQ ID NO: 36)

5′-aaagatcttgtgaatcaaatccttgtttaaatgg-3′

R primer:

(SEQ ID NO: 37)

5′-ttctcgagttaattcacagttccttccttcaaatc-3′

As a DNA encoding (iii) EGF 3rd of Del-1(D136E), a product obtained by introducing mutation into the above-described DNA encoding (ii) EGF 3rd of Del-1 obtained by means of PCR using QuikChange kit (Stratagene) was used.

Next, each of the expression vectors constructed as described above was introduced into a Cos1 cell to prepare a conditioned medium. As a negative control, only the pAPtag-4 vector was used. To a culture solution at the time of introduction of lacZ gene, a fusion protein of alkaline phosphatase and each of the above-described proteins was added, and to the negative control, alkaline phosphatase in an amount to have alkaline phosphatase activity equivalent to that of the above-described added fusion protein was added. 72 hours after the introduction of lacZ gene, the Pro5 cell was collected to measure β-galactosidase activity.

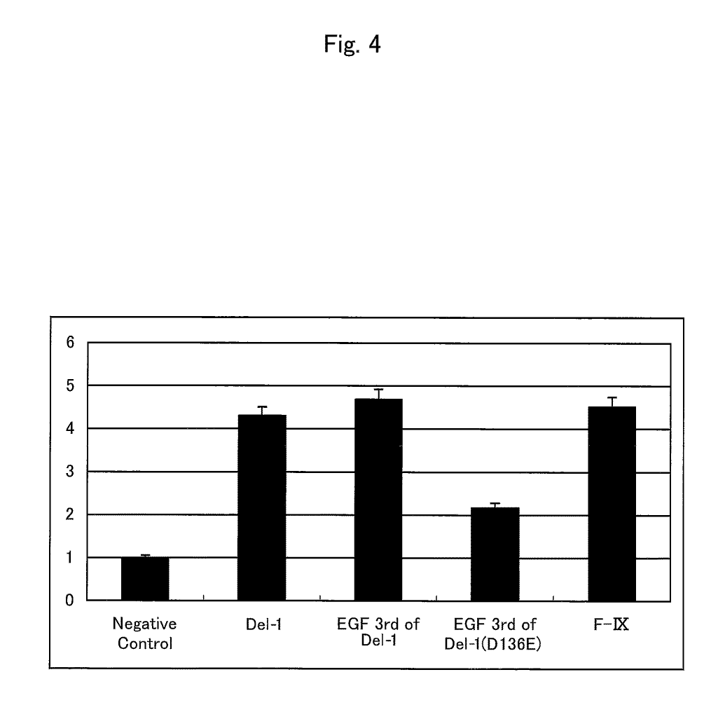

Results of the experiment are shown in FIG. 4.

Since EGF3 of Del-1 solely exerted the same effect as that of Del-1, it was thought that the active center exists in EGF3. Based on already-published reports regarding other types of proteins, it was predicted that aspartic acid at position 136 of the amino acid sequence of EGF3 would be affected by (3-hydroxylation. When aspartic acid in EGF3 was replaced by glutamic acid, the activity to increase the gene transfer efficiency was significantly suppressed. Thus, it was found that aspartic acid in EGF3 is an important amino acid. Further, EGF domain of coagulation factor IX, which is a naturally-occurring protein having a structure similar to that of EGF3 of Del-1, exerted high activity to increase the gene transfer efficiency as in the case of EGF3 of Del-1. Therefore, it was indicated that not only EGF3 of Del-1, but also family proteins comprising an amino acid sequence similar thereto have activity to increase the gene transfer efficiency.

### Example 5

### Effect of Promoting Gene Transfer by Del-1 In Vivo

Confirmation was made as to whether or not the peptide of the present invention has the effect of promoting gene transfer in vivo.

### (1) Method

According to the ordinary method, transgenic mice in which cDNA of full-length Del-1 (i.e., DNA consisting of the nucleotide sequence represented by SEQ ID NO: 9) is overexpressed were prepared (see Hidai et al., Anatomical Record A, vol. 287A, pp. 1165-1175). Del-1 protein produced in cells of the transgenic mice is a protein having the amino acid sequence represented by SEQ ID NO: 10 (including the signal peptide). Del-1 protein secreted from the cells to the outside thereof is a protein having an amino acid sequence consisting of amino acid residues at positions 26 to 480 in the amino acid sequence represented by SEQ ID NO: 10 (excluding the signal peptide). Using TransIT In Vivo Gene Delivery System (Minis), the above-described transgenic mice were subjected to tail vein injection of human thermostable alkaline phosphatase gene. 24 hours later, blood was collected from the mice and serum was separated. After separation, the collected serum was subjected to heat treatment at 65° C. for 30 minutes to inactivate endogenous alkaline phosphatase. Using 20 μl of serum after the heat treatment, the activity of exogenous alkaline phosphatase (human thermostable alkaline phosphatase) was measured. In the measurement of the activity, 20 μl of serum was mixed with 200 μl of 1 mg/ml p-nitrophenyl phosphate, and after causing reaction at room temperature for 30 minutes to 1 hour, the absorbance at 405 nm was measured using a micrometer (BioRad).

As a control group, wild-type mice born of the same mother as the transgenic mice were employed. The wild-type mice were also subjected to injection of human thermostable alkaline phosphatase gene in a manner similar to that in the case of the transgenic mice. After that, collecting blood and the like and heat treatment were carried out to measure exogenous alkaline phosphatase activity.

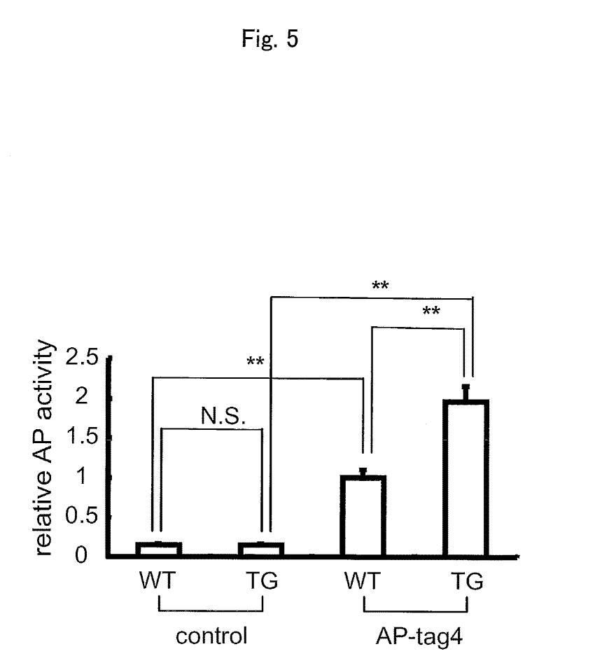

Measurement values of the alkaline phosphatase activity are expressed by average value±SEM (standard error of the mean). The measurement values obtained from the control group into which alkaline phosphatase gene was not injected are regarded as 1, and the measurement values of the transgenic mice were calculated based thereon. Results are shown in FIG. 5.

### (2) Results

Firstly, as shown by the left side (control) of the graph in FIG. 5, in the case where the human thermostable alkaline phosphatase gene was not injected into the mice, it was confirmed that there is no difference between the endogenous alkaline phosphatase activity of the wild-type mouse (WT) and that of the transgenic mouse (TG).

Next, as shown by the right side (AP-tag4) of the graph in FIG. 5, in the case where the human thermostable alkaline phosphatase gene was injected into the mice and collecting blood and the like and heat treatment were carried out as described above to measure exogenous alkaline phosphatase activity, the alkaline phosphatase activity of the transgenic mouse (TG) was about twice as high as that of the wild-type mouse (WT). Therefore, it was confirmed that the peptide of the present invention (full-length Del-1) increases the gene transfer efficiency of TransIT In Vivo Gene Delivery System.

### INDUSTRIAL APPLICABILITY

By utilizing the auxiliary reagent of the present invention, the gene transfer efficiency in various gene transfer methods can be significantly increased. The auxiliary reagent of the present invention can increase the gene transfer efficiency regardless of what kind of gene transfer method is used or what kind of host cell or transfer reagent is used, and therefore is excellent in terms of usefulness. Therefore, even in a cell in which only low transfer efficiency is realized by a commercially available gene transfer reagent, the transfer efficiency of a gene of interest can be dramatically increased, and accordingly, the efficiency of experiments, researches, etc. can be significantly improved.

### Sequence Listing Free Text

### SEQ ID NO: 1: Peptide

SEQ ID NO: 1: Xaa at Location: 3 represents aspartic acid (Asp) or asparagine (Asn), Xaa at Location: 8 represents phenylalanine (Phe) or tyrosine (Tyr), and Xaa at Locations: 2, 4, 5, 6, 7, 9 and 11 represents any amino acid residue.

### SEQ ID NO: 2: Peptide

SEQ ID NO: 2: Xaa at Locations: 2, 4, 5, 6, 7, 9 and 11 represents any amino acid residue.

### SEQ ID NO: 3: Peptide

SEQ ID NO: 3: Xaa at Locations: 2, 4, 5, 6, 7, 9 and 11 represents any amino acid residue.

### SEQ ID NO: 4: Peptide

SEQ ID NO: 4: Xaa at Locations: 2, 4, 5, 6, 7, 9 and 11 represents any amino acid residue.

### SEQ ID NO: 5: Peptide

SEQ ID NO: 5: Xaa at Locations: 2, 4, 5, 6, 7, 9 and 11 represents any amino acid residue.

### SEQ ID NO: 6: Peptide

SEQ ID NO: 6: Xaa at Location: 3 represents aspartic acid (Asp) or asparagine (Asn), Xaa at Location: 8 represents phenylalanine (Phe) or tyrosine (Tyr), and Xaa at
Locations: 2, 4, 5, 6, 7, 9 and 11 represents any amino acid residue.

### SEQ ID NO: 7: Peptide

SEQ ID NO: 7: Xaa at Location: 3 represents aspartic acid (Asp) or asparagine (Asn),
Xaa at Location: 8 represents phenylalanine (Phe) or tyrosine (Tyr), and Xaa at Locations: 2, 4, 5, 6, 7, 9 and 11 represents any amino acid residue.

### SEQ ID NO: 32: Synthetic DNA

### SEQ ID NO: 33: Synthetic DNA

### SEQ ID NO: 34: Synthetic DNA

### SEQ ID NO: 35: Synthetic DNA

### SEQ ID NO: 36: Synthetic DNA

SEQ ID NO: 37: Synthetic DNA

1-15. (canceled)

16. An auxiliary reagent for gene transfer comprising a peptide of (a) or (b) below, a derivative thereof, or a salt of the peptide or the derivative:
(a) a peptide comprising an amino acid sequence represented by the following formula (I):
C-X-[D/N]-X-X-X-X-[F/Y]-X-C-X-C (I)
wherein X represents any amino acid residue, C represents cysteine, [D/N] represents aspartic acid or asparagine, and [F/Y] represents phenylalanine or tyrosine;
(b) a peptide, which comprises the amino acid sequence represented by the formula (I) having deletion, substitution or addition of one or several amino acids, and which has activity to increase the efficiency of gene transfer into a host cell.

17. A DNA encoding a peptide of (a) or (b) below:
(a) a peptide comprising an amino acid sequence represented by the following formula (I):
C-X-[D/N]-X-X-X-X-[F/Y]-X-C-X-C (I)
wherein X represents any amino acid residue, C represents cysteine, [D/N] represents aspartic acid or asparagine, and [F/Y] represents phenylalanine or tyrosine;
(b) a peptide, which comprises the amino acid sequence represented by the formula (I) having deletion, substitution or addition of one or several amino acids, and which has activity to increase the efficiency of gene transfer into a host cell.

18. A recombinant vector comprising the DNA according to claim 17.

19. A transformant comprising the recombinant vector according to claim 18.

20. A gene transfer method, wherein a gene is introduced into a host cell in the presence of the reagent according to claim 16.

21. A kit for gene transfer, comprising the reagent according to claim 16.

22. An auxiliary reagent for gene transfer comprising a peptide of (a) or (b) below, a derivative thereof, or a salt of the peptide or the derivative:
(a) a peptide consisting of an amino acid sequence represented by SEQ ID NO: 10;
(b) a peptide, which consists of the above-described amino acid sequence in (a) having deletion, substitution or addition of one or several amino acids, and which has activity to increase the efficiency of gene transfer into a host cell.

23. An auxiliary reagent for gene transfer comprising a peptide of (a) or (b) below, a derivative thereof, or a salt of the peptide or the derivative:
(a) a peptide consisting of an amino acid sequence represented by SEQ ID NO: 12;
(b) a peptide, which consists of the above-described amino acid sequence in (a) having deletion, substitution or addition of one or several amino acids, and which has activity to increase the efficiency of gene transfer into a host cell.

24. An auxiliary reagent for gene transfer comprising a peptide of (a) or (b) below, a derivative thereof, or a salt of the peptide or the derivative:
(a) a peptide consisting of an amino acid sequence represented by SEQ ID NO: 30;
(b) a peptide, which consists of the above-described amino acid sequence in (a) having deletion, substitution or addition of one or several amino acids, and which has activity to increase the efficiency of gene transfer into a host cell.

25. A gene transfer method, wherein a gene is introduced into a host cell in the presence of the reagent according to any one of claims 22 to 24.

26. A kit for gene transfer, comprising the reagent according to any one of claims 22 to 24.

27. A DNA encoding a peptide of (a) or (b) below:
(a) a peptide consisting of an amino acid sequence represented by SEQ ID NO: 12;
(b) a peptide, which consists of the above-described amino acid sequence in (a) having deletion, substitution or addition of one or several amino acids, and which has activity to increase the efficiency of gene transfer into a host cell.

28. A DNA encoding a peptide of (a) or (b) below:
(a) a peptide consisting of an amino acid sequence represented by SEQ ID NO: 30;
(b) a peptide, which consists of the above-described amino acid sequence in (a) having deletion, substitution or addition of one or several amino acids, and which has activity to increase the efficiency of gene transfer into a host cell.

29. A recombinant vector comprising the DNA according to claim 27 or 28.

30. A transformant comprising the recombinant vector according to claim 29.
